(19) United States
(12) Patent Application Publication
(10) Pub. No.: US 2025/0327749 A1
(43) Pub. Date: Oct. 23, 2025

(54) Measuring Apparatus

(71) Applicant: HYDAC Filter Systems GmbH, Sulzbach / Saar (DE)

(72) Inventors: Jörg Steffensky, Dillingen (DE); Torsten Bley, Beckingen (DE); Philipp Götz, Eppelborn (DE)

(73) Assignee: HYDAC Filter Systems GmbH, Sulzbach / Saar (DE)

(21) Appl. No.: 18/864,306

(22) PCT Filed: Apr. 24, 2023
(86) PCT No.: PCT/EP2023/060682

§ 371 Date: Nov. 8, 2024

(30) Foreign Application Priority Data
May 9, 2022 (DE) 10 2022 111 450.3

(51) Int. Cl. G01N 21/41; G01N 21/01; G01N 33/26

(52) U.S. Cl. CPC G01N 21/4133; G01N 21/01; G01N 33/26

(57) ABSTRACT

Disclosed is a measuring apparatus for determining the concentration of constituents in a fluid, such as cooling lubricants or HFC hydraulic liquids, by refractometry, wherein the fluid present for measuring is guided through a sample chamber, which is connected to a fluid inlet and a fluid outlet and which is at least partially transparent such that the beams of a light source, for example in the form of a laser, passing through the sample chamber containing the fluid at least partially experience a refraction and can be detected by a sensor device outside the sample chamber.

### CROSS-REFERENCE TO RELATED APPLICATIONS

This application claims priority to German Patent Application DE 10 2022 111 450.3, filed on May 9, 2022 with the German Patent and Trademark Office. The contents of the aforesaid Patent Application are incorporated herein for all purposes.

### BACKGROUND

This background section is provided for the purpose of generally describing the context of the disclosure. Work of the presently named inventor(s), to the extent the work is described in this background section, as well as aspects of the description that may not otherwise qualify as prior art at the time of filing, are neither expressly nor impliedly admitted as prior art against the present disclosure.

The disclosure relates to a measuring apparatus for determining the concentration of constituents in a fluid, such as cooling lubricants or HFC hydraulic liquids, by refractometry.

DE 10 2010 028 319 A1 discloses a method for controlling the concentration of water-mixed cooling lubricant in a machine tool and an associated apparatus, which serve both to measure the index of refraction of the water-mixed cooling lubricant by refractometry and to measure the electrical conductivity of the water-mixed cooling lubricant, and to combine the values obtained from both measurements to give a control variable with which a top-up with water and/or cooling lubricant takes place if the control variable deviates from the target value. To determine the index of refraction of the water-mixed cooling lubricant, a digital refractometer is used in the apparatus, comprising an LED as the light source and a CCD sensor as the detector.

### SUMMARY

A need exists to provide an improved measuring apparatus with which one or more disturbance variables arising during the measurement can be compensated.

The need is addressed by the subject matter of the independent claim(s). Embodiments of the invention are described in the dependent claims, the following description, and the drawings.

### BRIEF DESCRIPTION OF THE DRAWINGS

FIG. 1 takes the form of a longitudinal section showing example components of the measuring apparatus;

FIGS. 2 to 5 show different options for carrying out measurements using the transmitted light principle;

FIGS. 6, 7, 8 and 10 show different types of operation of the example measuring apparatus using flowcharts; and

FIG. 9 takes the form of an example hydraulic circuit diagram showing how the measuring apparatus is incorporated in a hydraulic measurement and supply circuit.

### DESCRIPTION

The details of one or more embodiments are set forth in the accompanying drawings and the description below. Other features will be apparent from the description, drawings, and from the claims.

In the following description of embodiments of the invention, specific details are described in order to provide a thorough understanding of the invention. However, it will be apparent to one of ordinary skill in the art that the invention may be practiced without these specific details. In other instances, well-known features have not been described in detail to avoid unnecessarily complicating the instant description.

In some embodiments, the fluid present for measuring is guided through a sample chamber, which is connected to a fluid inlet and a fluid outlet and which is at least partially transparent such that the beams of a light source, for example in the form of a laser, passing through the sample chamber containing the fluid at least partially experiences a refraction and can be detected by a sensor device outside the sample chamber. This leads to a spatial separation of the light source, sample chamber and sensor device, and a number of adjustment and correction options arise in this manner such that the measuring apparatus can be used for a wide range of measurement tasks and calibrated for these accordingly. As the fluid to be tested is guided via a sample chamber, the sample is thus also disconnected from the actual measuring device, consisting of the light source and the sensor device, such that can be carried out in an undisrupted manner measurements independently of the actual supply circuit for a hydraulic load. For example, a light source in the form of a laser is used in this case, which, unlike the customary LED technology which is otherwise used, permits collimation, i.e., leads to a parallel alignment of otherwise divergent light beams, which results in an improved measured value resolution on the part of the sensor device, which is usually formed by a photodiode array or diode array.

The laser also allows a higher radiant flux to be applied, which means that reliable measured value detection is always provided even if the fluid is cloudy and/or the sample chamber, part of which is kept transparent, happens to be contaminated.

In some embodiments of the measuring apparatus, it is provided that the sample chamber is delimited on its side facing the sensor device by a translucent wall, for example in the form of a glass wall, and the light source is received in a receiving chamber of an apparatus housing and fluid flows over said light source at least partially before entering the sample chamber. It has proved to be beneficial if there is turbulence in the fluid flow when flowing through the sample chamber. This is important in order to clean contamination of the glass wall of the sample chamber and, in any event, to replace the sample fluid in the measuring or sample chamber in each instance.

In this case, it is for example provided that light is emitted from the light source at an oblique angle, for example 40°, to the fluid flow direction in the sample chamber, and that the two-dimensional extension of the sensor device and its position with respect to the light source are selected such that, in the event of transmitted light, the light beams hitting the sensor device at different angles are detected. In this manner, without the need to make major changes to the measurement structure, the refractometer can be operated with transmitted light referred to the light source according to the relevant measurement task.

In some embodiments of the measuring apparatus, it is provided that the sensor device is part of a sensor chamber of a sensor housing, which is filled with a gas, for example with air, and creates a spatial distance between the sample chamber with its translucent wall and the sensor surface of the sensor device. For example, in this case, viewed in a notional vertical projection, the light source is arranged at the start of the sample chamber and the beginning of the sensor device is arranged at the end of the sample chamber. Then, in any event, by selecting the aforementioned spatial 1 distance and the respective selected projection plane for the arrangement of the sensor device, this can be adjusted both in the vertical and the horizontal direction so as to adjust the sensitivity and/or the measurement range in this manner.

In order to ensure a for example turbulent fluid flow, it is provided that a fluid channel with individual channel portions runs at least partially between the fluid inlet and the fluid outlet in a supply housing such that a turbulent flow through the sample chamber comes about as a result of multiple deflection.

In some embodiments of the measuring apparatus, it is provided that the entire housing of the apparatus is composed of individual housing parts, consisting of the supply housing containing parts of the fluid channel, the apparatus housing containing the light source and the sensor housing containing the sensor device. The measuring apparatus can quickly be dismantled and reassembled for maintenance and cleaning purposes due to the multi-housing-part structure. A form of modular structure is also achieved in this manner for the entire housing, which, in practice, makes it easier to retrofit hydraulic devices that have already been delivered and are in operation with the measuring apparatus according to the teachings herein.

In some embodiments of the measuring apparatus, it is provided that said apparatus is connected via a switchable valve to a pressure supply device, such as a hydraulic pump, which takes its fluid from a storage tank, which hydraulically supplies machining equipment as a load, said machining equipment being connected with its inlet side via a branch to a fluid line between the hydraulic pump and the switchable valve, and that the outlet side of the machining equipment emerges into a return line at a branch point, which is connected to the fluid outlet in the supply housing and leads to the storage tank. In this manner, the measuring apparatus in the secondary branch can be disconnected from the actual pressure supply for the hydraulic load such that measurements can be carried out at discrete time intervals outside operation of the hydraulic load. For example, for this purpose, it is provided that a further switching valve is accommodated in the portion of the return line between the fluid outlet in the supply housing and the branch point into which the outlet side of the machining equipment emerges.

In some embodiments of the measuring apparatus, it is provided that a third and a fourth switching valve is in each case connected to the supply line to the fluid inlet and to the return line from the fluid outlet, said switching valves serving to supply or remove a flushing medium. In this manner, in turn, independently of operation of the machining equipment, in the event of contamination arising in the measuring apparatus, this can be removed by means of a flushing operation.

Reference will now be made to the drawings in which the various elements of embodiments will be given numerical designations and in which further embodiments will be discussed.

Specific references to components, process steps, and other elements are not intended to be limiting. Further, it is understood that like parts bear the same or similar reference numerals when referring to alternate FIGS. The FIGS. are schematic and not necessarily to scale.

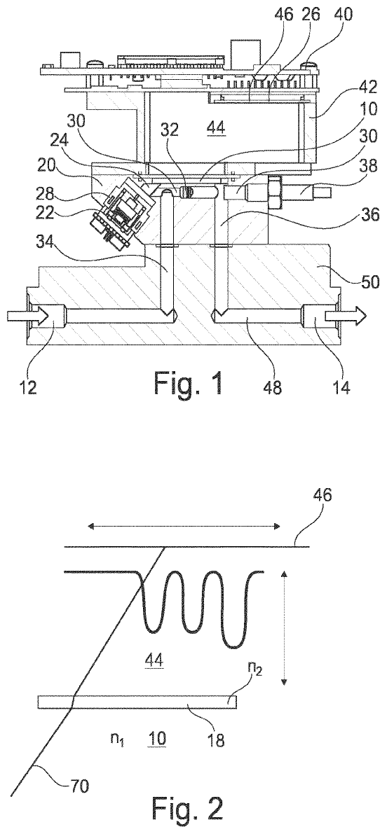

The measuring apparatus shown in FIG. 1 with system components is shown in the usual operating position. The measuring apparatus serves to determine the concentration of constituents in a fluid, such as cooling lubricants or HFC hydraulic fluids, by refractometry. Hydraulic fluid is generally used to transmit energy in the form of volume flow and/or pressure in hydraulic systems in the field of fluid technology. Corresponding hydraulic oils are usually manufactured based on mineral oil with corresponding additives. HFC is a fire-resistant hydraulic fluid and generally comprises water glycols with a water content in excess of 35% and a polyglycol solution. Corresponding HFC hydraulic fluids are regularly provided for use in coal mining and in the civil aviation industry. Furthermore, these are increasingly used in military vehicles such as tanks, which may be exposed to enemy fire. Cooling lubricants or cooling lubricant materials reduce friction due to lubrication and thus reduce wear on the tool, overheating of the tool and the energy required during cutting-type machining operations. In both cases, the proposed concentration of HFC and cooling lubricant should be maintained in order to provide reliable operation. As such, the measuring apparatus according to the teachings herein is used to maintain the respective concentration.

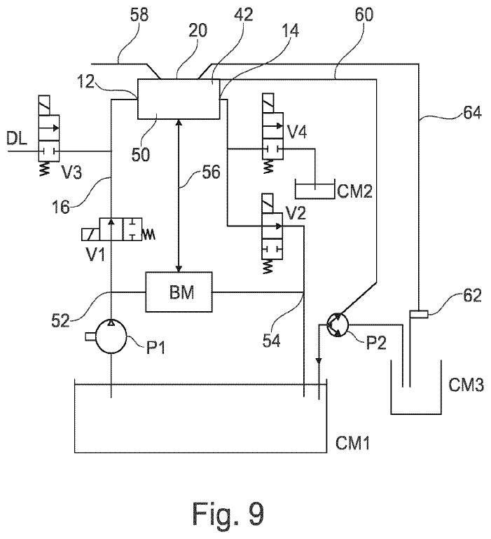

The fluid provided for measurement purposes by means of the measuring apparatus is guided through a sample chamber 10 which is connected to a fluid inlet 12 and a fluid outlet 14. In this case, the possible throughflow direction is shown in FIG. 1 with arrows at the inlet 12 and at the outlet 14. Both the fluid inlet 12 and the fluid outlet 14 are connected in the usual manner to a fluid supply circuit 16, as reproduced in FIG. 9 by way of example.

The actual sample chamber 10 delimits a cuboid chamber volume with a flat extension and, in the viewing direction seen on FIG. 1, is delimited from the top by a translucent glass wall 18; usually formed by a thin-walled rectangular glass pane, which is delimited towards the top and bottom on its outer circumference from adjacent housing parts of the measuring apparatus by square sealing rings so as to thus reliably avoid undesirable leakage of fluid from the sample chamber 10 into the environment. Abutting the side of the sample chamber 10, a laser 22 is fitted in an apparatus housing 20 of the measuring apparatus, the upper discharge surface of said laser emerging in a fluid-conveying oblique channel 24 in the direction of the sample chamber 10. In this manner, the beams from a light source, in this case in the form of the laser 22, pass through the sample chamber 10 containing the respective fluid and the corresponding beams thus experience a first refraction n, which will be explained in more detail below. The beams refracted in this manner are detected by a sensor device 26 outside the sample chamber 10. The sensor device 26 comprises a photodiode array, which is also referred to in technical jargon as a diode array, as a light-sensitive sensor. In particular, CCD sensors, but also CMOS sensors, are used in this respect, which, as light-sensitive electronic components, are based on the internal photo effect and are commercially available on the market in a range of embodiments.

As is also shown on FIG. 1, the light source in the form of the laser 22 is received in a stationary manner in an assigned receiving chamber 28 at one end of the apparatus housing 20 such that, before the fluid enters the actual sample chamber 10, the fluid flows over the discharge cross-section for the laser beams, in which said fluid flows from a horizontally extending pipe portion 30 parallel to the longitudinal orientation of the sample chamber 10 into the oblique channel 24. In the viewing direction seen on FIG. 1, the pipe portion 30 is sealed on its right-hand side by a plug 32, and otherwise pipe portions 34 and 36, which run vertically from the bottom emerge into the corresponding horizontal pipe portion 30, which, in the viewing direction seen on FIG. 1, continues onwards towards the right behind the plug 32 and emerges into the vertical pipe portion 36, to which the fluid outlet 14 is connected. However, the fluid inlet 12 leads from the left into the vertical pipe portion 34 for the fluid supply to the sample chamber 10. Beyond the vertical pipe portion 36, the horizontal pipe portion 30 is guided further towards the right and is sealed by a sensor 38 at this point, said sensor possibly being formed, by way of example, by a measuring device for parameters such as pressure, temperature, viscosity, pH value, conductivity, etc. Sensors 38, which allow two or more different parameter measurements of this kind to take place, may also be used in this case. In particular, a temperature measurement is required for temperature compensation in connection with refractometry.

As is also shown in FIG. 1, the light from the light source in the form of the laser 22 is emitted at an oblique angle of approximately 40° to the horizontally running fluid flow direction into the sample chamber 10. The rectangular two-dimensional extension of the sensor device 26 is in any event selected, with regard to its position with respect to the light source, such that, both in the event of the transmitted light method favoured here and in the event of any glancing incidence of light beams at different angles, these are detected, for example over the full circumference, by the sensor device 26. For the purpose of calibrating the measuring apparatus and in particular for adjusting the sensor device 26 to the actual measurement conditions inside the measuring apparatus, said apparatus may be adjusted, as shown in the drawing in FIG. 2, both horizontally and vertically with respect to the light discharge point on the laser 22. To this end, it is sufficient to loosen and then re-tighten screws on an adjustment device 40 to which the sensor device 26 is fixed and by means of which said device can be positioned with respect to a sensor housing 42 arranged in a stationary manner. As such, the sensor housing 42, as part of the overall housing, abuts the upper side of the apparatus housing 20. In particular, the plate-shaped sensor device 26 emerges into a square sensor chamber 44 of the sensor housing 42, which can be furnished with a gas and in this manner fills a spatial distance between the sample chamber 10 with its translucent wall 18 and an exposed sensor surface 46 of the sensor device 26. For ease of illustration, in FIG. 1, both the laser 22 and the sensors in the form of the device 26 and the respective measurement device 38 are shown without any associated wiring. According to the respective wavelength and within which index of refraction the sensor device 26 is to be loaded, a working gas other than air can also be received in the sensor chamber 44, for example in the form of xenon. In a notional, vertical projection within the drawing plane of FIG. 1, the light source in the form of the laser 22 is arranged at the start of the sample chamber 10 and the beginning of the sensor device 26 is arranged at the end of the sample chamber 10. In this manner, this leads to measured values being detected particularly well in the entire region and, due to the oblique position of the laser 22, this leads to a good diffraction image or interference pattern during irradiation of the fluid in the sample chamber 10 and, furthermore, due to the oblique angle of incidence of the laser beams on the sensor surface 46, the installation space for the sensor housing 42 and thus for the measuring apparatus as a whole can be minimised, with the result that a corresponding measuring apparatus can be accommodated even in restricted installation conditions. This also makes it easier to retrofit existing systems with the measuring apparatus.

The channel portions 34, 36 running between the fluid inlet 12 and the fluid outlet 14 thus at least partially form a fluid channel 48 in a supply housing 50. Accordingly, the entire housing of the apparatus is composed of individual housing parts, consisting in particular of the supply housing 50 containing parts of the fluid channel 48, the apparatus housing 20 containing the light source, in this case in the form of the laser 22, and the sensor housing 42 containing the sensor device 26. This thus results in a modular structure for the entire housing of the measuring apparatus, which allows the measuring apparatus to be connected to a wide variety of machines and apparatus parts by adjusting individual components.

As already mentioned at the outset, the measuring apparatus is part of a fluid supply circuit 16 and this can be connected via a switchable valve V1 to a pressure supply device such as a hydraulic pump P1. The correspondingly motor-driven hydraulic pump P1 takes fluid, such as cooling lubricant or HFC fluid, from a storage tank CM1 and hydraulically supplies customary machining equipment BM as a load. The corresponding machining equipment BM is connected on its inlet side via a branch 52 to a fluid line between the hydraulic pump P1 and the switchable valve V1. The outlet side of the machining equipment BM in turn emerges, at a branch point 54, into a return line, which is connected to the fluid discharge in the form of the fluid outlet 14 in the supply housing 50 of the measuring apparatus and leads to the storage tank CM1. A further switching valve V2 is provided in the aforementioned portion of the return line between the fluid outlet 14 in the supply housing 50 and the branch point 54 into which the outlet side of the machining equipment BM emerges. Furthermore, a third V3 and a fourth switching valve V4 is in each case connected to the supply line to the fluid inlet 12 and to the return line from the fluid outlet 14, said switching valves serving to supply or respectively remove a flushing medium DL into/from a further storage tank CM2.

A control line 56, which serves to transmit measurement data and allows a flushing operation to take place according to the status of the machine and/or measuring apparatus, runs between the machining equipment BM and the measuring apparatus, the housing of which is reproduced in FIG. 10 with housing parts 20, 42 and 50. Measurement parameter detection, which is at least partially performed via the sensor 38, transmits its measurement data via an additional measurement line 56 to a processor controller 59 as the higher-level system, which is not shown in further detail, as illustrated in FIG. 10. In addition to the usual measurement values of pressure, temperature and viscosity, it is also possible to detect the pH value of the fluid and its electrical conductivity via the sensor 38 or further sensors 1, 2, . . . x, which are not shown. Starting from a further control line 60 according to FIG. 9, the measuring apparatus is able to control a further fluid pump P2, which, if necessary, extracts missing concentrate detected by means of the measuring apparatus from a concentrate vessel CM3, the filling level in the concentrate vessel CM3 being monitored by a level switch 62, which is connected by means of a further measurement line 64 to the processor controller 59 of the measuring apparatus. Accordingly, if, in conjunction with the refractometry performed by means of the measuring apparatus, it is observed that lubricant constituents in connection with the coolant lubricant supply for the machining equipment BM are missing, or HFC in connection with the supply of an HFC hydraulic fluid is missing, the corresponding missing constituents can be added to the storage tank CM1 by actuating the supply pump P2 via the concentrate vessel CM3, and the resulting correctly concentrated cooling lubricant quantity or HFC hydraulic fluid then passes into the machining equipment BM, whereupon a refractometer measurement is accordingly continuously performed by means of the measuring apparatus as part of the concentration process. The adjustment means referred to as external actuator 1, 2, . . . in FIG. 10 in this case correspond inter alia to components P1 and P2 and to valves V1, V2, V3, V4 etc.

In the event of contamination, especially with regard to the sample chamber 10, the supply circuit 16 can be shut off by means of the valves V1, V2 and by opening the valves V3 and V4 the sample chamber 10 can be flushed by supplying an appropriate flushing medium DL including compressed air and, in this manner, cleaned of particulate contamination, which is then received in the storage tank CM2 for further treatment or disposal. After carrying out the flushing operation, the valves V3 and V4 can then be reset, actuated by spring force, to their original position as shown in FIG. 9, i.e., moved into their closing position, and, after opening the valves V1 and V2 again by switching on the fluid supply circuit 16, the measuring apparatus is then once again available for refractometry measurement.

The measuring apparatus is explained in further detail below with the aid of the associated measurement method. FIG. 2 accordingly shows such a measurement according to the transmitted light principle. In this method, the laser 22 shown in FIG. 1 emits a collimated laser beam 70, which experiences a first refraction n1 at the interface between the sample chamber 10 and the plate-like glass wall 18. A second refraction n2 then takes place at the glass wall 18 in the form of a standard glass pane. FIG. 2 describes the change in signal of the line array or sensor surface 46 respectively with different liquid concentrations. If a vertical adjustment takes place in which the vertical distance between the sensor surface 46 and the glass wall 28 is modified, this makes it possible to adjust the sensitivity. The measured value range can be adjusted by a possible horizontal displacement of the sensor surface 46. In the outline representation of the measured value detection process with the illustrated curve characteristic shown in FIG. 2, a homogeneous fluid is analysed in the sample chamber 10; however, it is also possible to inspect cloudy fluids. In principle, this is possible as the laser diode or the laser 22 respectively can be controlled with variable intensity by means of an open and/or closed-loop control device, which is not shown in greater detail, in the form of the processor control 59. For example, however, the laser 22 controls the intensity independently.

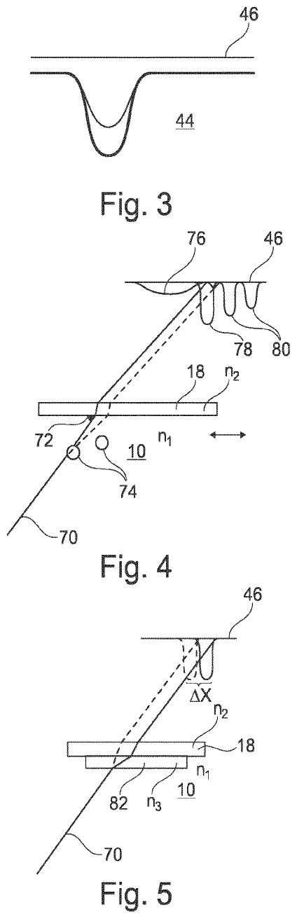

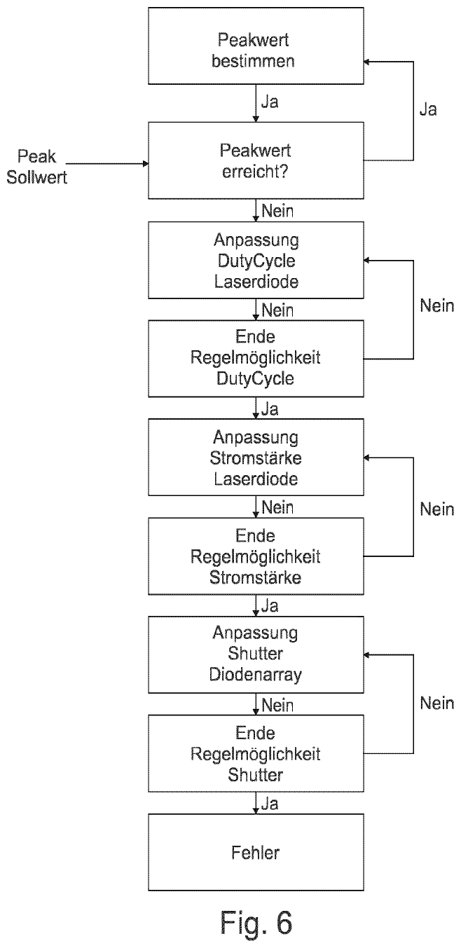

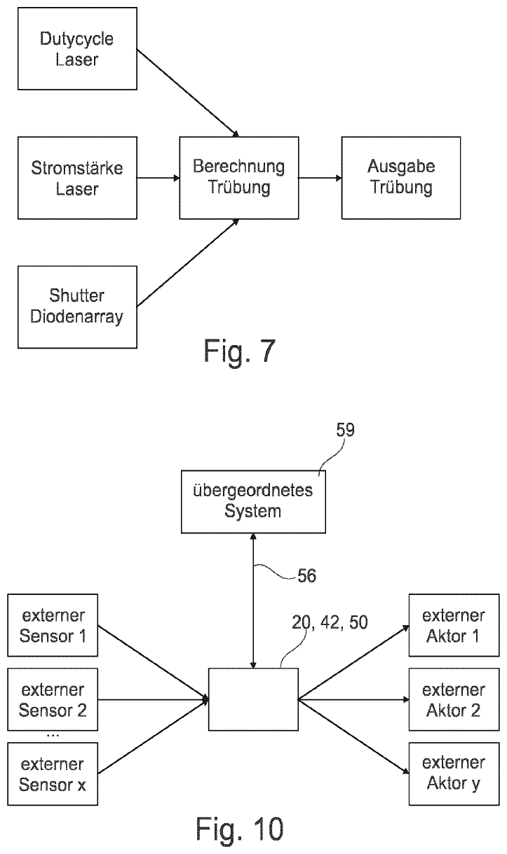

This kind of measured value curve caused by cloudiness of the fluid in the sample chamber 10 is reproduced by way of example in FIG. 3, the measured value curve shown in bold illustrating the original measured value curve and the non-bold measurement line relating to the loss of intensity due to cloudiness of the fluid. In order to return to the previous peak value recording once again despite the loss of intensity, as shown by the bold curve in FIG. 3, an adjustment of the laser 22 is required, for example by adjusting the power cycle or duty cycle, as it is known in technical jargon, or even by increasing the current intensity for the laser diode. A further adjustment option entails changing the frame rate or image refresh rate respectively, also referred to in technical jargon as shutter frequency, on the photodiode array or diode array in the form of the sensor device 26. A corresponding adjustment of the laser intensity or detector sensitivity respectively with regard to the occurrence of potential cloudiness of the fluid in the sample chamber 10 is reproduced by way of example in the flowchart shown on FIG. 6. In order to carry out the corresponding adjustment cycle, it is in any event a prerequisite that a peak value determination should be carried out as a reference, i.e., specifying a peak value for the light received by refraction on the diode array in the form of the sensor device 26 using a fluid to be analysed homogeneously in the sample chamber 10 as shown in FIG. 2. The actual cloudiness calculation incorporating output values is achieved as shown in FIG. 7 by recording measurement variables with regard to the duty cycle and the current intensity for the laser 20 including calculating the shutter frequency of the diode array of the sensor device 26. In this manner, the disturbance variables arising due to cloudiness can be compensated as part of the standard measurement.

In addition to the aforementioned cloudiness, as shown in FIG. 4, contamination may also arise in the fluid in the sample chamber 10 as a further potential disturbance variable, for example in the form of finely dispersed particles 72, such as those that may regularly arise in emulsions or in the form of larger particles 74 including air bubbles in the volume flow moved inside the sample chamber 10. In this case, the measured value curve that results from a broad increase in intensity due to laser scattering as caused by the finely dispersed particles 72 in the fluid flow is shown to the far right of FIG. 4, based on an average peak value 78 with a rounded measurement value curve as obtained during the usual refraction by fluid n1 and glass pane n2. The short-term sharp peaks with variable intensity that arise from a different refraction caused by the aforementioned particles 74 or the incorporation of air bubbles are significantly different from the above. The corresponding disturbance variables can also be compensated as they are detected individually and do not disrupt the concentrate determination of the fluid used with the measuring apparatus.

The drawing in FIG. 5 in turn relates to a different disturbance variable in connection with the concentration measurement, in which a translucent, surface contamination with a different refraction value n3 arises on the glass wall 18 with its refraction value of n2. Accordingly, FIG. 5 shows the peak curve on the sensor surface 46 (diode array) as a dashed line, without contamination 82 and the right-hand curve shows the evaluation with the contamination applied to the glass pane 18. Accordingly, the two peak curves shown in FIG. 5 with the same measured value levels, viewed in the horizontal direction, are displaced by a value of Ax, which can be evaluated and thus permits conclusions as to the level of contamination 42 on the glass pane 18. As such, this disturbance variable can then also be calculated again when determining the fluid concentrate.

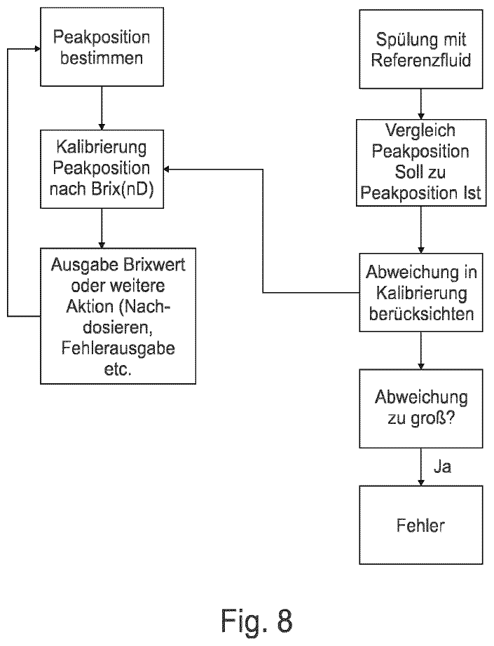

In the case of all the aforementioned disturbance variables, such as fluid cloudiness, particulate contamination or impurities on the glass wall 18, as described above for FIG. 9, a flushing operation can be performed for the sample chamber 10, the associated procedure being reproduced in outline in FIG. 8. In this case, a measurement and flushing process can basically take place as follows:

OPEN=Supply the measuring apparatus by opening valves V1 and V2

MEAS=Measurement 1 is performed for a defined period (fluid=cooling lubricant or HFC)

CLOSE=Stop the flow to the measuring apparatus by closing valve V1 and valve V2

FL1=Start flushing operation by opening valves V3 and V4

FLU=Flush the sample chamber 10 for a defined period

FL2=End the flushing operation, valves V3 and V4 remain open

CAL=Measurement 2 is performed for a defined period (fluid=flushing fluid, water or through air)

EVAL=Evaluation of measurement 2 from CAL and reporting (measurement OK, recalibration, servicing required)

FL3=Close valves V3 and V4

The refractometer described above to measure the concentration of the concentrate of a cooling lubricant or an HFC liquid or other fluids where the concentration of constituents needs to be monitored, carries out individual discrete measurements, during which the index of refraction to determine the concentration of cooling lubricant lies between 0 and 25% Brix (value of the index of refraction) and that of HFC lies between 30 and 50% Brix. As part of self-diagnostics, it is possible to carry out a regular internal check on the sensor device 26 to determine the validity of the measurement data. If, for example, no peak values (hotspots) can be detected on the diode array or sensor surface 46 respectively due to excessive cloudiness in the fluid, the sensor device 26 should not issue any further measured values and this should be displayed by the status of the sensor device 26.

Furthermore, what is known as an in-line calibration can be carried out using the measuring apparatus. After flushing the sample chamber 10 or measurement cell respectively, a reference measurement is performed in water or air respectively. If a deviation from the expected value of the flushing fluid is measured, the sensor device 26 is automatically recalibrated. To this end, the measured value with flushing fluid is used as the new zero value. Furthermore, a ‘Clean refractometer’ or similar warning is issued. By evaluating the deviation from the original value when starting up, it is also possible to predict when the laser 22 and/or the glass wall 18 will need to be exchanged based on damage to the glass pane in accordance with the embodiments shown on FIGS. 2 to 5. This therefore has no parallel in the prior art.

The invention has been described in the preceding using various exemplary embodiments. Other variations to the disclosed embodiments may be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor, device, or other unit may be arranged to fulfil the functions of several items recited in the claims. Likewise, multiple processors, devices, or other units may be arranged to fulfil the function of several items recited in the claims.

The term “exemplary” used throughout the specification means “serving as an example, instance, or exemplification” and does not mean “preferred” or “having advantages” over other embodiments. The term “in particular” and “particularly” used throughout the specification means “for example” or “for instance”.

The mere fact that certain measures are recited in mutually different dependent claims or embodiments does not indicate that a combination of these measures cannot be used to advantage. Any reference signs in the claims should not be construed as limiting the scope.

What is claimed is:

1-10. (canceled)

11. A measuring apparatus for determining the concentration of constituents in a fluid, such as cooling lubricants or HFC hydraulic liquids, by refractometry, wherein
the fluid present for measuring is guided through a sample chamber, which is connected to a fluid inlet and a fluid outlet and which is at least partially transparent such that the beams of a light source passing through the sample chamber containing the fluid at least partially experience a refraction and can be detected by a sensor outside the sample chamber.

12. The measuring apparatus of claim 11, wherein
the sample chamber is delimited on its side facing the sensor by a translucent wall; and wherein
the light source is received in a receiving chamber of an apparatus housing and fluid flows at least partially over said light source before entering the sample chamber.

13. The measuring apparatus of claim 11, wherein
light is emitted from the light source at an oblique angle to the fluid flow direction in the sample chamber; and wherein
a two-dimensional extension of the sensor and its position with respect to the light source are selected such that, in the event of transmitted light and in the event of eventual glancing incidence of light beams at different angles, these are detected by the sensor.

14. The measuring apparatus of claim 11, wherein the sensor is part of a sensor chamber of a sensor housing, which is filled with a gas, and creates a spatial distance between the sample chamber with its translucent wall and the sensor surface of the sensor.

15. The measuring apparatus of claim 11, wherein, viewed in a notional vertical projection, the light source is arranged at the start of the sample chamber and the beginning of the sensor is arranged at the end of the sample chamber.

16. The measuring apparatus of claim 11, wherein a fluid channel with individual channel portions runs at least partially between the fluid inlet and the fluid outlet in a supply housing.

17. The measuring apparatus of claim 11, wherein an entire housing of the apparatus is composed of individual housing parts, consisting of the supply housing containing parts of the fluid channel, the apparatus housing containing the light source and the sensor housing containing the sensor.

18. The measuring apparatus of claim 11, wherein said apparatus is connected via a switchable valve to a pressure supply device, such as a hydraulic pump, which takes its fluid from a storage tank, which hydraulically supplies machining equipment as a load, said machining equipment being connected with its inlet side via a branch to a fluid line between the hydraulic pump and the switchable valve, and wherein the outlet side of the machine equipment emerges into a return line at a branch point, which is connected to the fluid outlet in the supply housing and leads to the storage tank.

19. The measuring apparatus of claim 11, wherein a further switching valve is connected in the portion of the return line between the fluid outlet in the supply housing and the branch point into which the outlet side of the machine equipment emerges.

20. The measuring apparatus of claim 11, wherein a third and a fourth switching valve is in each case connected to the supply line to the fluid inlet and to the return line from the fluid outlet, said switching valves serving to supply or remove a flushing medium.

21. The measuring apparatus of claim 11, wherein the light source comprises a laser.

22. The measuring apparatus of claim 12, wherein the translucent wall is in the form of a glass wall.

23. The measuring apparatus of claim 12, wherein
light is emitted from the light source at an oblique angle to the fluid flow direction in the sample chamber; and wherein
a two-dimensional extension of the sensor and its position with respect to the light source are selected such that, in the event of transmitted light and in the event of eventual glancing incidence of light beams at different angles, these are detected by the sensor.

24. The measuring apparatus of claim 12, wherein the sensor is part of a sensor chamber of a sensor housing, which is filled with a gas, and creates a spatial distance between the sample chamber with its translucent wall and the sensor surface of the sensor.

25. The measuring apparatus of claim 31, wherein the sensor is part of a sensor chamber of a sensor housing, which is filled with a gas, and creates a spatial distance between the sample chamber with its translucent wall and the sensor surface of the sensor.

26. The measuring apparatus of claim 12, wherein, viewed in a notional vertical projection, the light source is arranged at the start of the sample chamber and the beginning of the sensor is arranged at the end of the sample chamber.

27. The measuring apparatus of claim 13, wherein, viewed in a notional vertical projection, the light source is arranged at the start of the sample chamber and the beginning of the sensor is arranged at the end of the sample chamber.

28. The measuring apparatus of claim 14, wherein, viewed in a notional vertical projection, the light source is arranged at the start of the sample chamber and the beginning of the sensor is arranged at the end of the sample chamber.

29. The measuring apparatus of claim 12, wherein a fluid channel with individual channel portions runs at least partially between the fluid inlet and the fluid outlet in a supply housing.

30. The measuring apparatus of claim 13, wherein a fluid channel with individual channel portions runs at least partially between the fluid inlet and the fluid outlet in a supply housing.
